(19) United States
(12) Patent Application Publication
(10) Pub. No.: US 2025/0170624 A1
(43) Pub. Date: May 29, 2025

(54) CLEANING UNIT FOR CLEANING A HOUSING OF A MEDICAL IMAGING DEVICE THAT SURROUNDS A PATIENT-ACCOMMODATING REGION

(71) Applicant: Hans Georg HAGLEITNER, Zell am See (AT)

(72) Inventor: Hans Georg HAGLEITNER, Zell am See (AT)

(21) Appl. No.: 18/958,158

(22) Filed: Nov. 25, 2024

(30) Foreign Application Priority Data
Nov. 24, 2023 (AT) A 50949/2023

(51) Int. Cl. B08B 9/00; A61B 5/00; A61B 5/055; A61B 6/00; A61B 6/03

(52) U.S. Cl. CPC B08B 9/00; A61B 5/0033; A61B 6/4423; A61B 5/055; A61B 6/032; A61B 2560/06

(57) ABSTRACT

A cleaning unit for cleaning a housing of a medical imaging device that surrounds a patient-accommodating region, having a support structure and having a cleaning component, the cleaning component of the cleaning unit introduced into the housing being arranged between the support structure and the housing. In a pressure medium-filled state, the cleaning component, in a pressure medium-filled state, rests against the housing, preferably with contact pressure.

### CROSS-REFERENCE TO RELATED APPLICATION

This application claims the priority, under 35 U.S.C. § 119, of Austrian Patent Application A 50949/2023, filed Nov. 24, 2023; the prior application is herewith incorporated by reference in its entirety.

### FIELD AND BACKGROUND OF THE INVENTION

The present invention relates to a cleaning unit for cleaning a housing of a medical imaging device that surrounds a patient-accommodating region, having a support structure and having a cleaning component. The invention further pertains to a medical imaging device with such a cleaning unit, and to a method for cleaning such a medical imaging device.

Medical imaging devices are used in radiology and include MRI and CT equipment, for example (MRI—magnetic resonance imaging; CT—computed tomography). The devices are dirtied during use and/or contaminated with germs and/or viruses. For example, within the medical imaging device, this can be caused by the persons to be examined themselves, should germs or other contamination be left behind following coughing, sneezing, humid respiration, physical contact with the medical imaging device, or the like. Such contamination remains, inter alia, on the inner side of the medical imaging device, specifically on a housing.

In this case, manual cleaning is brought about using a cleaning utensil that is optionally secured to a rod, for example as disclosed in U.S. Pat. No. 5,918,342.

A disadvantage thereof is that this type of cleaning is very laborious and time consuming, and/or the cleaning staff is located very close or sometimes even within the medical imaging device. The radiological influence by the medical imaging device, for example x-ray radiation or magnetic fields, can be uncomfortable in that context.

The provision of automated cleaning units within the medical imaging device is also known, for example, as disclosed in the published Chinese utility model CN 219251060 U or patent application US 2023/0090506 A1(corresponds to German published patent application DE 10 2021 210 471 A1). In that case, the cleaning unit can perform wiping-type cleaning, by virtue of the cleaning unit being displaced from one end of the housing to the other end of the housing.

A disadvantage thereof is that a cleaning unit of this type, following a first displacement of the cleaning unit and wiping of the housing, at least partly recontaminates the housing as the cleaning unit moves back into the starting position since the germs and/or viruses picked up during the first displacement may be left behind again on the housing. In addition, in the current prior art, when a cleaning unit of this type is introduced into the housing of a medical imaging device, the positioning and the geometrical accuracy of the cleaning unit has to be either very precise or a complicated mechanism to compensate for tolerances has to be provided. Deviations and/or inaccuracies in this respect have a negative effect on the cleaning performance.

### SUMMARY OF THE INVENTION

The problem to be solved by the present invention thus consists in rectifying the disadvantages of the prior art at least in part, and in specifying an improved cleaning unit over the prior art, which is distinguished in particular by reliable cleaning of medical imaging devices. It is a further object of the invention to provide for a medical imaging device having such a cleaning unit and a method for cleaning such a medical imaging device wi.

With the above and other objects in view there is provided, in accordance with the invention, a cleaning unit for cleaning a housing of a medical imaging device that surrounds a patient-accommodating region, the cleaning unit comprising:

a support structure and a cleaning component;

when the cleaning unit is introduced into the housing, said cleaning component being arranged between said support structure and the housing; and

said cleaning component, in a pressure medium-filled state, resting against and contacting the housing.

Alternatively, or in addition, the cleaning unit comprises a support structure and a cleaning component, the cleaning component is arranged along an outer contour of the support structure; and the cleaning component is deformable by filling with a pressure medium.

In other words, the objects of the invention are achieved, by way of example, by means of a cleaning unit for cleaning a housing of a medical imaging device that surrounds a patient-accommodating region, having a support structure and having a cleaning component, the cleaning component of the cleaning unit introduced into the housing being arranged between the support structure and the housing, wherein the cleaning component, in a pressure medium-filled state, rests against the housing, preferably with contact pressure.

In the current context, the term “cleaning” is understood to mean cleansing, disinfecting, and/or sterilizing. For example, cleaning can be implemented in the form of wiping, abrading, scraping, rubbing, scrubbing, washing, and/or the like.

By means of a pressure medium-filled state of the cleaning components, resting of the cleaning component against the housing is ensured. In a non-filled state, the cleaning component does not rest against the housing. The pressure of the pressure medium with which the cleaning component is filled makes it possible to control the resting pressure. A lower pressure of the pressure medium leads to the resting pressure being weak, whereas a higher pressure of the pressure medium leads to the resting pressure being greater.

The above objects are also achieved, according to a second aspect of the invention, by means of a cleaning unit for cleaning a housing of a medical imaging device that surrounds a patient-accommodating region, having a support structure and having a cleaning component, the cleaning component being arranged along a, preferably outer, contour of the support structure, wherein the cleaning component is deformable by filling with a pressure medium.

Here, the term “deformable” is understood to mean that the shape of a body can be modified as a consequence of the action of a force and/or a stress. A deformation of a body can be broken down into a first component, in which the volume and/or the spatial extent of the body changes, but not the geometric shape of the body, for example in the event of an increase in the radius of a sphere or a tubular body, and into a second component, in which the geometric shape of the body changes, but not the volume and/or the spatial extent of the body, for example in the event of a sphere being stretched along one axis to form an ellipsoid. In the event of a deformation as a result of the cleaning component being filled with a pressure medium, there can be a deformation with a first and/or a second component.

Here, the expression “cleaning component deformable by filling with a pressure medium” can be understood to mean that a cleaning component is deformable by virtue of a volume determined in part, preferably in full, by the cleaning component being taken up at least in part, preferably in full, by a pressure medium.

Deformability of the cleaning component as a result of filling with a pressure medium causes a modification of a geometric shape, preferably an external diameter, of the spatial extent and/or the volume of the cleaning component. Contact between the cleaning unit and the housing of the medical imaging device can be brought about or omitted in a targeted manner in this way.

By means of the two above-outlined aspects of the invention, reliable cleaning of a housing can be performed by virtue of the cleaning component being put into a filled state, the housing being contacted by the filled cleaning component and the cleaning component cleaning the housing, for example by wiping, when the cleaning unit is moved. As soon as the moving of the cleaning unit along the housing in a first direction is ended, the filled cleaning component can be put into a non-filled state and the cleaning unit with the non-filled cleaning component can be moved contactlessly back again into the starting position. No renewed contact of the cleaning component with the housing occurs, and therefore recontamination is avoided.

A “non-filled cleaning component” or a “cleaning component in a non-filled state” is a cleaning component which is not filled with a pressure medium, wherein the non-filled cleaning component of a cleaning unit introduced into the housing does not come into contact with the housing.

In a non-filled state, the cleaning component is affected by force and/or stress due to the atmospheric pressure surrounding the cleaning component and optionally due to a medium, not the pressure medium, present in the cleaning component, but said cleaning component is by no means affected by pressure from the housing.

A “partly filled cleaning component” or a “cleaning component in a partly filled state” is a cleaning component which is filled at least in part with a pressure medium, wherein the partly filled cleaning component of a cleaning unit introduced into the housing does not come into contact with the housing.

A “filled cleaning component” or a “cleaning component in a filled state” is a cleaning component which is filled with a pressure medium, wherein the filled cleaning component of a cleaning unit introduced into the housing comes into contact with the housing.

In a filled state, the cleaning component is affected by force and/or stress due to the pressure medium, due to the atmospheric pressure surrounding the cleaning component and due to the housing in contact with the cleaning component and optionally by a reactive force of the cleaning component, the preferably elastic cleaning component, as a consequence of the deformation.

In the transition from a filled cleaning component, in which there is contact with the housing, via a partly filled cleaning component to a non-filled cleaning component, pressure medium is removed from the cleaning component, whereby a spatial extent of the cleaning component is reduced, and contact with the housing is terminated.

In the transition from a non-filled cleaning component, in which there is no contact with the housing, via a partly filled cleaning component to a filled cleaning component, pressure medium is introduced into the cleaning component, whereby a spatial extent of the cleaning component is increased, and contact with the housing is achieved.

Only once a sufficient amount of pressure medium has been introduced into a cleaning component of a cleaning unit or, in other words, only once the cleaning component has been sufficiently filled with pressure medium does the transition occur from the partly filled cleaning component to the filled cleaning component, whereby contact with the housing by way of a cleaning unit introduced into the housing is made possible. Should an already filled cleaning component be subsequently filled even more with pressure medium, there is pressurized contact with the housing, wherein the contact pressure is determined by the degree of filling of the filled cleaning component.

A cleaning component can be filled a plurality of times in succession and increase its spatial extent according to the explanations given above every time it is refilled. This applies analogously to a cleaning component from which some of the pressure medium is taken a plurality of times in succession.

A further advantage is that the filled cleaning component can close all of the gaps between the cleaning unit and the housing, and therefore gap-free cleaning, for example wiping, can be made possible.

Further advantages are:
A uniform contact pressure of the filled cleaning component against the housing can be realized.
Manufacturing tolerances of the housing and the positioning of the displacement device, for example a movable patient table and/or a patient table guide to and/or within the housing can be compensated for.
Different dimensions, preferably internal diameters, of various housings of different models of medical imaging devices can be compensated for.
Simple provision of a gap between the cleaning component and housing during the movement of the cleaning component after cleaning of the housing.
Preparation work or maintenance work, e.g. changing cleaning units or individual parts of the cleaning unit, can always be carried out from the same side of the medical imaging devices.

For the sake of completeness, attention is drawn to the fact that the numerals used within the description of this invention, for instance one, two, three, and the like, as a matter of principle only describe the minimum quantity present of a feature of the cleaning unit according to the invention, of the medical imaging device according to the invention having such a cleaning unit and/or of the method according to the invention for cleaning such a medical imaging device. It is self-evident that individual features or constituent parts can also be present in a greater quantity. For example, the cleaning unit according to the invention may include more than one cleaning component, more than one support structure, etc. In this sense, the numeral one, for example, should thus advantageously be understood in the sense of at least one, etc.

According to a preferred embodiment of the cleaning unit, provision is made for an outer contour of the support structure and/or of the, preferably filled, cleaning component to preferably be curved, particularly preferably in a circular manner, corresponding to an inner contour of the housing.

In a preferred embodiment variant, provision can be made for an outer contour of the non-filled cleaning component to correspond to an inner contour of the housing, with a gap being provided between the contours.

In other words, if one contour corresponds to another contour, the one contour follows the other contour.

Gap-free contacting can be achieved even more reliably by the mutually corresponding and preferably curved, particularly preferably circular, contours of the support structure and/or of the cleaning component and the housing since the filled cleaning component scarcely has to compensate for deviations in shape.

In a preferred embodiment variant, provision can be made for the support structure to be dimensionally stable and/or for the cleaning component to be elastically deformable.

A higher pressure of the pressure medium and hence a greater spatial extent can be attained by way of the elastic deformability of the cleaning component. The cleaning component can therefore be adapted even better and more flexibly to the housing and/or pressed more tightly thereagainst.

In a preferred embodiment variant, provision can be made for the support structure and/or the cleaning component to contain or consist of at least one non-metallic material, preferably wherein the at least one non-metallic material contains or consists of at least one of the following materials:
natural materials, particularly preferably graphite,
polymer materials, particularly preferably elastomers, thermoplastics, thermosets, copolymers,
inorganic materials, particularly preferably ceramics or glass,
composites, particularly preferably reinforced fiber composites or laminate composites.

By avoiding metallic materials in the cleaning unit, negative effects of a magnetic field to the medical imaging device, e.g. damage to the equipment, risk of injury, higher outlay on installation, maintenance, etc., can be avoided or at least reduced.

According to a preferred embodiment of the cleaning unit, provision is made for a spatial extent, preferably an external diameter, of the cleaning unit
to be smaller in the event of a non-filled cleaning component than the spatial extent, preferably the external diameter, of the cleaning unit with a filled cleaning component, and/or
to be smaller in the event of a non-filled cleaning component than a spatial extent, preferably an internal diameter, of the housing, and/or
to be at least as large or larger in the event of a filled cleaning component as/than a spatial extent, preferably an internal diameter, of the housing.

According to a preferred embodiment of the cleaning unit, provision can be made for the pressure medium to be a gas or a liquid, preferably compressed air.

A gas or a liquid as pressure medium can afford different advantages. For example, gases are compressible and can therefore better compensate pressures that are too high. Liquids are hardly compressible and therefore transmit pressures virtually without pressure loss.

According to a preferred embodiment of the cleaning unit, provision is made for a deformation device to be provided, wherein the deformation device comprises a deformation drive, a pressure medium and a connecting line for connecting, preferably detachably or non-detachably connecting, the cleaning component to the deformation drive, wherein the pressure medium can be filled into the cleaning component and/or removed from the cleaning component by means of the deformation drive, wherein the cleaning component is deformable by filling with the pressure medium and/or removing the pressure medium.

The expressions “detachable connection,” “detachably connected,” and “detachably connectable” can be understood to mean that at least two initially separate components can be connected to one another, wherein the detachable connection can subsequently be non-destructively released, with the result that the at least two components are again present in their original separated state, in order to be able to be subsequently connected again. “Detachable connection,” “detachably connected,” and “detachably connectable” can therefore also be referred to as “non-destructively detachable connection,” “non-destructively detachably connected,” and “non-destructively detachably connectable”.

“Non-detachable connection,” “non-detachably connected,” and “non-detachably connectable” can be understood to mean that at least two initially separate components can be connected to one another, wherein this non-detachable connection cannot be subsequently released without the integrity of at least one of the at least two components being damaged and/or without a renewed connection of the at least two components being made more difficult or being precluded. “Non-detachable connection,” “non-detachably connected,” and “non-detachably connectable” can therefore also be referred to as “non-destructively non-detachable connection,” “non-destructively non-detachably connected,” and “non-destructively non-detachably connectable.”

In a preferred embodiment variant, provision can be made for a deformation device to comprise a compressor, preferably an air compressor, wherein the compressor aspirates the pressure medium, preferably ambient air, guides the latter under pressure to the cleaning component via a connecting line and thus fills the cleaning component.

An open-loop and closed-loop control device can be used for open-loop control and/or closed-loop control of the amount of pressure medium and/or the pressure to be attained within the filled cleaning component.

At least one sensor, preferably a pressure sensor, can be provided in order to monitor the state of the filled and/or non-filled cleaning component.

An open-loop and closed-loop control device can be connected to sensors.

According to a preferred embodiment of the cleaning unit, provision is made for the cleaning component to have a cleaning main body and a cleaning covering, preferably wherein the cleaning covering is placed along an outer contour of the cleaning main body.

In a preferred embodiment variant, provision can be made for the cleaning main body to be able to be a tubular body.

In a preferred embodiment variant, provision can be made for the cleaning main body to be able to be a flat material web, preferably a membrane, wherein at least one end, preferably at least two ends, of the flat material web, preferably of the membrane, can be connected to the support structure, preferably along an outer contour of the support structure.

In a preferred embodiment variant, provision can be made for the cleaning main body to be able to be an extruded profile and/or an extruded double profile.

In a preferred embodiment variant, provision can be made for the cleaning covering to comprise a foam and/or a textile structure and/or a nonwoven material.

In a preferred embodiment variant, provision can be made for the cleaning covering to comprise a sponge and/or a nonwoven fabric.

In a preferred embodiment variant, provision can be made for the cleaning covering to comprise or consist of a squeegee lip.

In a preferred embodiment variant, provision can be made for the cleaning covering to be moistened or unmoistened.

According to a preferred embodiment of the cleaning unit, provision is made for a placement device to be provided, wherein the cleaning covering can be placed, preferably can be stretched, on the cleaning main body, preferably along an outer contour of the cleaning main body, by means of the placement device.

In a preferred embodiment variant, provision can be made for the placement device to comprise a placement drive and a connecting device for the connection, preferably the detachable or non-detachable connection, to the cleaning covering, wherein the cleaning covering connected by means of the connecting device can be moved on the cleaning main body, preferably by being unrolled from a roll, by means of the placement drive until an operating position is reached.

The placement device makes it possible for the placing, preferably stretching, of the cleaning covering to be at least partly automated, as a result of which the outlay on maintenance of the cleaning unit can be reduced and/or more reliable maintenance can be achieved.

In an operating position of the cleaning covering, the cleaning covering is arranged on the cleaning component in such a manner that the cleaning unit is ready for operation and can be used for cleaning the housing. Provision can preferably be made for the cleaning covering to partly or completely cover the cleaning main body in the operating position.

The placement device can be used to remove a cleaning covering from the cleaning unit or, in other words, to displace a cleaning covering of the cleaning unit. Hence, a preferred embodiment variant can include a removal of a first, preferably used, cleaning covering and/or a placement of a second, preferably unused, cleaning covering.

According to a preferred embodiment of the cleaning unit, provision is made for an application device for applying a cleaning product to or into the cleaning covering to be provided, wherein the cleaning product can be applied by means of the application device before, during and/or after the placement of the cleaning covering on the cleaning main body.

Here, a “cleaning product” can be understood to mean a well-known product for cleansing purposes, for example soap, a disinfectant, for example alcohol, and/or a sterilization means.

The application device makes it possible for cleaning product to be applied to and/or introduced into the cleaning covering in an at least partly automated manner, as a result of which the outlay on maintenance of the cleaning unit can be reduced and/or more reliable maintenance can be achieved. In addition, greater flexibility of the cleaning and greater user friendliness can thus be achieved. For example, by selection of an appropriate application device, the time for moistening a nonwoven cloth within a cleaning process can be defined. In addition or instead, moistening of a nonwoven cloth repeatedly is possible.

According to a preferred embodiment of the cleaning unit, provision is made for the cleaning unit to be movable within the medical imaging device by means of a displacement device, preferably wherein the displacement device is a movable patient table of the medical imaging device or a displacement device which is separate from the medical imaging device.

In the case in which the movable patient table is the displacement device, the advantage consists in that the patient table is an already present displacement device of a medical imaging device and, by means of the coupling to the cleaning unit, permits a further function, specifically the cleaning of the housing.

In a preferred embodiment variant, provision can be made for the cleaning unit to be connectable or connected, preferably detachably or non-detachably, to the displacement device. With the aid of a coupling device for the connection, preferably the detachable or non-detachable connection, of the cleaning unit to the displacement device, the cleaning unit can be moved by means of the displacement device into the housing and/or within the housing and/or out of the housing. After or during the detachment of the cleaning unit from the displacement device, the cleaning unit can be deposited within the housing or at or in the vicinity of an opening of the patient-accommodating region.

In a preferred embodiment variant, provision can be made for at least one further cleaning component to be provided, preferably wherein the at least one further cleaning component is arranged, particularly preferably directly, adjacent to the cleaning component.

In a preferred embodiment variant, provision can be made for the at least one further cleaning component to be arranged along a, preferably outer, contour of the support structure or along a, preferably outer, contour of at least one further support structure.

In the event that the further cleaning component is arranged on the same support structure as the cleaning component, the width of the support structure can be adapted correspondingly to two or more cleaning components.

A further cleaning component or, in other words, at least two cleaning components of a single cleaning unit affords/afford the advantage that the cleaning action is improved.

In this connection, it can be provided, for example, that, in the event of heavy soiling, two cleaning components can be used simultaneously. On the other hand, it can also be provided that the housing can be cleaned in a first direction with a first cleaning component and can be cleaned in a second direction, specifically in the direction opposed to the first direction and leading back to the starting position, with a second cleaning component. The housing can thus be cleaned twice directly in succession without a cleaning covering having to be changed after a first cleaning operation or one of the cleaning coverings recontaminating the housing.

Furthermore, protection is sought for a medical imaging device having a scanner unit, a patient-accommodating region at least partly surrounded by the scanner unit, a housing that surrounds the patient-accommodating region, and having at least one cleaning unit according to the invention, wherein the cleaning unit is movable within the housing.

According to a preferred embodiment of the medical imaging device, provision is made for

the non-filled cleaning component to be surrounded substantially, preferably completely, contactlessly by the housing, and

for the filled cleaning component to rest against the housing, preferably without a gap, particularly preferably with contact pressure.

According to a preferred embodiment of the medical imaging device, provision is made for the cleaning unit to be connected or connectable, preferably non-detachably or detachably, to a displacement device, preferably to a movable patient table of the medical imaging device.

In a preferred embodiment variant of the medical imaging device, provision can be made for the cleaning unit to be movable into the housing and/or movable out of the housing and/or movable in at least one direction, preferably two directions, within the housing by means of the displacement device.

A displacement device of this type can be used to automate at least a movement of the cleaning unit. In a particularly preferred embodiment variant, provision can be made for the cleaning unit to be moved in an at least partly, preferably fully, automated manner from a parking position into the housing where the cleaning of the housing is carried out and then for the cleaning unit to be moved back into the parking position. The outlay on cleaning the housing is thus reduced.

An open-loop and closed-loop control device can be provided for moving the cleaning unit.

Furthermore, protection is sought for a method for cleaning a medical imaging device according to the invention with a cleaning unit according to the invention, comprising the following method steps:

providing the cleaning unit with a filled cleaning component and

cleaning a housing of the medical imaging device by means of the filled cleaning component.

According to a preferred embodiment of the method for cleaning, provision is made for the method to comprise at least one of the following method steps:

moving the cleaning unit into the housing,

cleaning the housing by means of the filled cleaning component along a first direction and/or along a second direction,

providing the cleaning unit with a non-filled cleaning component,

contactlessly moving the non-filled cleaning component within the housing, preferably in a first direction and/or a second direction,

moving the cleaning unit out of the housing.

In a preferred embodiment variant of the method for cleaning,

provision can be made for the method to include at least one, or several, but preferably all, of the following method steps:

providing the cleaning unit with a filled cleaning component and with a further non-filled cleaning component,

cleaning the housing along a first direction by means of the filled cleaning component,

contactlessly moving the further non-filled cleaning component in a first direction within the housing,

providing the cleaning unit with the non-filled cleaning component and with the further filled cleaning component,

contactlessly moving the non-filled cleaning component in a second direction within the housing,

cleaning the housing along a second direction by means of the further filled cleaning component.

This preferred exemplary embodiment of a method can make it possible, as already described above, to use at least two cleaning components within a cleaning cycle, wherein the at least two cleaning components can be used simultaneously or in succession without a cleaning covering having to be changed or there being the risk of recontamination.

In a preferred embodiment variant of the method for cleaning, provision can be made for the method to comprise at least one, preferably all, of the following method steps:

providing the cleaning unit with a filled cleaning components,

constricting a, preferably filled, cleaning component, preferably along an outer contour of the cleaning component, and/or wherein the course of the constriction is inclined with respect to a first direction, particularly preferably is orthogonal, in order to provide a filled cleaning component with at least two contact surfaces, wherein at least one of the at least two contact surfaces rests against the housing, preferably with contact pressure, when the cleaning component is filled,

cleaning the housing along a first direction by means of at least one of the at least two contact surfaces of the filled and constricted cleaning component,

providing the cleaning unit with the non-filled cleaning component,

contactlessly moving the non-filled cleaning component in a second direction within the housing.

This preferred exemplary embodiment of a method can make it possible for at least two cleaning steps to take place during the movement of the cleaning unit in a first direction within the housing without recontamination occurring.

For example, the constriction of an individual cleaning component, specifically of a membrane, which is connected to the support structure, as cleaning main body and of a nonwoven fabric, which is arranged on the membrane, as cleaning covering, can lead to the cleaning component forming two beads. These two beads can run along an outer contour of the cleaning component, and the course of the beads can be oriented in an inclined manner, preferably orthogonally, to a direction of movement of the cleaning unit within the housing. In this way, a first bead can carry out preliminary cleaning in a first direction, while the second bead carries out subsequent cleaning directly afterwards in the first direction. When the cleaning unit terminates the movement and therefore the cleaning in the first direction, the cleaning component can be put into the non-filled state, as a result of which the two beads of the cleaning component are no longer in contact with the housing and can be moved back in the second direction.

It can be provided that when a cleaning component is filled and constricted and has at least two contact surfaces, at least one contact surface makes contact with the housing and at least one contact surface does not make contact with the housing.

In this way, during the movement of the cleaning unit in a first direction, the housing can be cleaned with at least one first contact surface, while at least one second contact surface does not make contact with the housing. Subsequently, it can be provided that the filling and/or the constriction of the cleaning component are/is manipulated in such a manner that, during the movement of the cleaning unit in a second direction, the housing is cleaned with the at least one second contact surface, while the at least one first contact surface no longer makes contact with the housing.

In a preferred embodiment variant, provision can be made for cleaning of a housing of the medical imaging device to be additionally provided by means of an irradiation device, preferably using UV radiation.

The combination of cleaning a housing of the medical imaging device using the filled cleaning component and an irradiation device can enable even more reliable cleaning.

Other features which are considered as characteristic for the invention are set forth in the appended claims.

Although the invention is illustrated and described herein as embodied in a cleaning unit for cleaning a housing of a medical imaging device that surrounds a patient-accommodating region, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a schematic, perspective view of a medical imaging device;

FIG. 2 is a schematic, perspective view into the housing of the medical imaging device from FIG. 1 with a cleaning unit according to the invention;

FIG. 3 is a schematic, perspective view of a first embodiment variant of the cleaning unit from FIG. 2;

FIG. 4 shows a schematic, perspective view of a second embodiment variant of the cleaning unit from FIG. 2;

FIG. 5 is a front view of the cleaning unit of FIG. 3;

FIG. 6 is a sectional view, taken along the line A-A in FIG. 5, of the cleaning unit in a non-filled state;

FIG. 7 shows a front view of the cleaning unit from FIG. 3 in a filled state;

FIG. 8 is a sectional view, taken along the line A-A in FIG. 7, of the cleaning unit in a filled state;

FIG. 9 shows various sectional illustrations A-A of the cleaning unit of FIG. 3 with different degrees of filling with the pressure medium;

FIGS. 10 to 12 show various front views of a housing with the cleaning unit of FIG. 3 arranged therein in a filled or non-filled state.

### DETAILED DESCRIPTION OF THE INVENTION

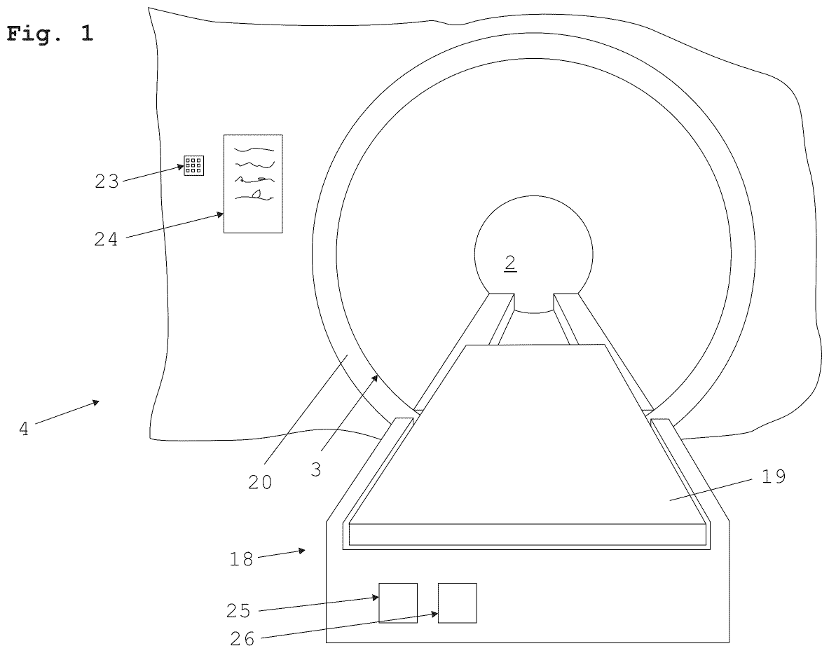

Referring now to the figures of the drawing in detail and first, in particular, to FIG. 1 thereof, there is shown a schematic, perspective view of a medical imaging device 4.

The medical imaging device 4 in FIG. 1 is a medical imaging device 4 having a scanner unit 20, a patient-accommodating region 2 at least partly surrounded by the scanner unit 20, and a housing 3 that surrounds the patient-accommodating region 2.

The medical imaging device 4 can comprise at least one cleaning unit 1 according to the invention, wherein the cleaning unit 1 can be movable within the housing 3.

The medical imaging device 4 can comprise an operating device 23 and an output device 24.

The medical imaging device 4 can comprise a displacement device 18. The displacement device 18 can comprise a movable patient table 19, a displacement drive 25 and an open-loop and closed-loop control unit 26.

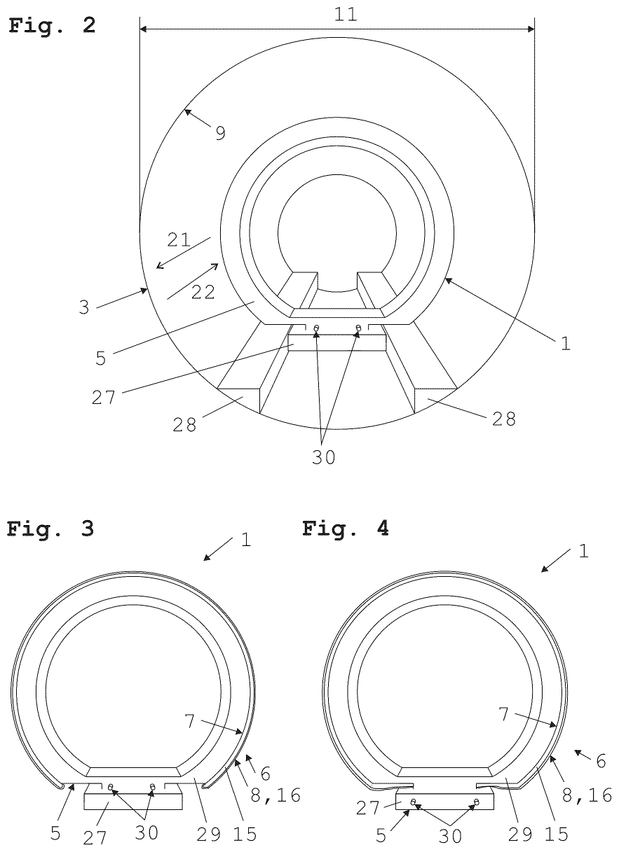

FIG. 2 shows a schematic, perspective view into the housing 3 of the medical imaging device 4 from FIG. 1 with a cleaning unit 1 according to the invention.

FIG. 2 shows a cleaning unit 1 for cleaning a housing 3 of a medical imaging device 4 that surrounds a patient-accommodating region 2, having a support structure 5 and having a cleaning component 6, the cleaning component 6 of the cleaning unit 1 introduced into the housing 3 being arranged between the support structure 5 and the housing 3, characterized in that the cleaning component 6, in a pressure medium-filled state, rests against the housing 3, preferably with contact pressure.

The cleaning component 6 has been left out in FIG. 2 for reasons of clarity.

The support structure 5 of the cleaning unit 1 can comprise a base part 27 and a ring part 29. However, it is also conceivable for the support structure not to have a base part 27.

The cleaning unit can comprise at least one coupling device 30 for the connection, preferably detachable or non-detachable connection, to a displacement device 18.

At least one coupling device 30 can be provided on the support structure, preferably on the base part 27 and/or on the ring part 29.

The medical imaging device 4 can have two light strips 28.

The housing 3 can have an inner contour 9. In the case of a hollow-cylindrical housing 3, as illustrated in FIG. 2, the inner side of the housing 3 corresponds to a circumferential surface of a cylinder. An inner contour 9 of the housing can thus be circular or arc-shaped.

The cleaning unit 1 can be moved within the housing 3 in at least one direction, preferably in a first direction 21 and/or in a second direction 22.

FIG. 3 shows a schematic, perspective view of a first embodiment variant of the cleaning unit 1 from FIG. 2.

The cleaning unit 1 can comprise a support structure 5 and a cleaning component 6.

The support structure 5 can comprise a base part 27 and a ring part 29, wherein the base part 27 can comprise a coupling device 30 for the connection, preferably detachable or non-detachable connection, to a displacement device 18.

The ring part 29 of the support structure 5 in FIGS. 3 and 4 has an outer contour 7 which describes virtually a complete circle, specifically an arc having an angle of more than 180°. Provision can be made for the support structure 5 to have an outer contour 7 which has a complete circle or an incomplete circle, specifically an arc.

The cleaning component 6 can comprise a cleaning main body 15 and a cleaning covering 16, wherein the cleaning main body 15 can be arranged between the support structure 5, specifically the ring part 29 and/or the outer contour of the support structure 5, and the cleaning covering 16.

The cleaning covering 16 can have a flexible embodiment such that the cleaning covering 16 adapts to the shape of the cleaning main body 15. In FIGS. 3 and 4, the shape of an outer contour of the cleaning main body 15 substantially corresponds to the shape of the outer contour of the cleaning covering 16 arranged on the cleaning main body 15.

In FIGS. 3 and 4, the shape of an outer contour 7 of the support structure 5 substantially corresponds to the shape of the outer contour 8 of the cleaning component 6, specifically in FIGS. 3 and 4 the outer contour of the cleaning covering 16 arranged on the cleaning component 6.

In FIGS. 3 and 4, the shape of the outer contour 8 of the cleaning component 6 is substantially circular, specifically arc-shaped.

FIG. 4 shows a schematic, perspective view of a second embodiment variant of the cleaning unit 1 from FIG. 2.

The above information stated with reference to FIG. 3 applies analogously to FIG. 4.

FIG. 3 and FIG. 4 differ to the extent
that the cleaning component 6 in FIG. 4 runs beyond the arc of the ring part 29 as far as the base part 27, and
that the coupling devices 30 in FIG. 4 are arranged on the base part 27 instead of on the ring part 29.

FIG. 4 continues with the cleaning covering 16 and the cleaning main bodies 15.

In this way, both the inner side of the housing 3 and also at least one side of the light strips 28, specifically the upper side of the light strip 28, can be cleaned.

The position of the coupling devices 30 can be provided on the base part 27 and/or on the ring part 29. In the situations in which the displacement device 18 for moving the cleaning unit 1 is the patient table of the medical imaging device or in which the displacement device is a device which is separate from the medical imaging device, different positions of the coupling device may be advantageous.

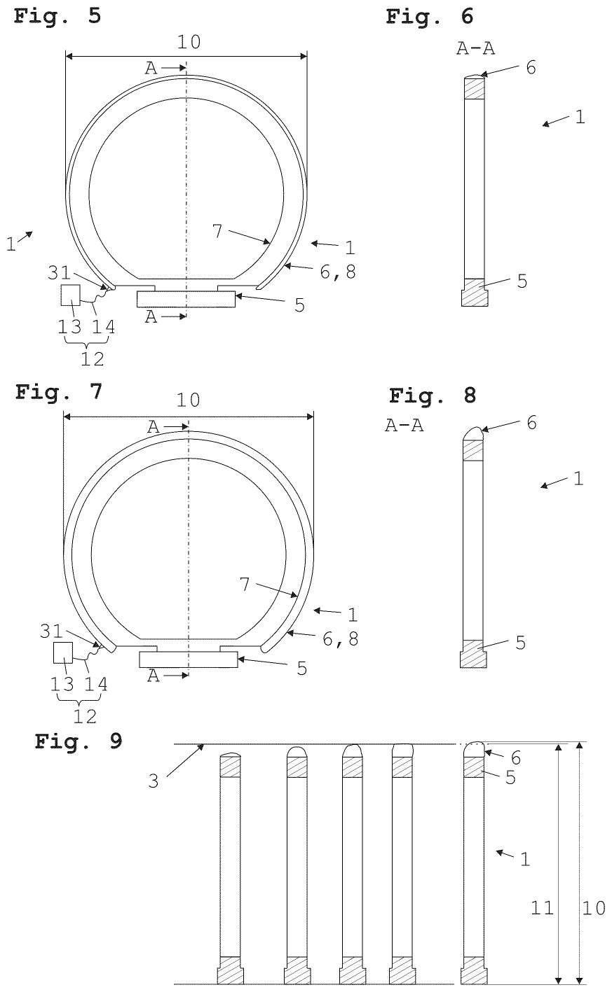

For the rest of the embodiments in FIGS. 5 to 12, the cleaning covering 16 and the coupling devices 30 are no longer explicitly illustrated for reasons of clarity. The cleaning covering 16 and the coupling devices 30 can nevertheless be provided.

For the remaining embodiments in FIGS. 5 to 12, the cleaning component 6 already described from FIG. 3 is used, with the cleaning component 6 described in FIG. 4 being able to be used in addition and/or instead.

It should be noted at this juncture that the cleaning unit 1 may have at least two identical cleaning components 6 or at least two different cleaning components, for example the cleaning component 6 from FIG. 3 and the cleaning component from FIG. 4.

FIG. 5 shows a front view of the cleaning unit 1 from FIG. 3 in a non-filled state.

In FIG. 5, the cleaning component 6 contains only little pressure medium, if any at all. It is important that the outer contour 8 of the cleaning component 6 has a spatial extent 10 which is smaller than a spatial extent 10 of the cleaning component 6 in the filled state and/or smaller than a spatial extent 11 of the housing 3. In other words, it is important that the outer contour 8 of the cleaning component 6 in the non-filled state does not make contact with the housing.

A deformation device 12 can be provided, wherein the deformation device 12 has a deformation drive 13 and a connecting line 14. The connecting line 14 serves for connecting the deformation drive 13 to the cleaning unit 1, specifically to the cleaning component 6. The cleaning unit 1 can have a connector 31 for the connecting line 14. The connector can be closable and opened and/or closed either manually or automatically.

The deformation drive 13 can be, for example, an air compressor which conveys air from the surroundings through the connecting line 14 and thus fills the cleaning component 6 with compressed air.

FIG. 6 shows a sectional illustration A-A of the cleaning unit 1 from FIG. 3 in a non-filled state.

It is clearly apparent in FIG. 6 that the cleaning component 6 encloses little to no pressure medium at all.

FIG. 7 shows a front view of the cleaning unit from FIG. 3 in a filled state.

It can clearly be seen in FIG. 7 that the spatial extent 10 of the cleaning component 6 is larger than the spatial extent 10 of the cleaning component 6 in FIG. 5. This filled state of the cleaning component 6 can be achieved by filling the cleaning component 6 with a pressure medium by means of the deformation device 12.

FIG. 8 shows a sectional illustration A-A of the cleaning unit from FIG. 3 in a filled state.

It is clearly apparent in FIG. 8 that the cleaning component 6 encloses more pressure medium than in FIG. 6.

FIG. 9 shows various sectional illustrations A-A of the cleaning unit 1 from FIG. 3 with different degrees of filling with the pressure medium.

The different degrees of filling of the cleaning component 6 are illustrated from the left to the right in FIG. 9, with the degree of the filling increasing from the left to the right.

The first illustration in FIG. 9 shows a cleaning component 6 in the non-filled state and the second illustration of a cleaning component 6 with a small amount of pressure medium, which is enclosed by the cleaning component 6, shows the cleaning component 6 in the partially filled state. In none of these two illustrations of a non-filled or partially filled cleaning component does the cleaning component 6 make contact with the housing 3.

The third illustration in FIG. 9 shows a first filled state of the cleaning component 6, in which the filled cleaning component 6 makes contact with the housing 3.

The fourth illustration in FIG. 9 shows a second filled state of the cleaning component 6, with more pressure medium being enclosed by the cleaning component 6 in the fourth illustration than in the third illustration. The contact surface of the cleaning component 6 of the fourth illustration with the housing 3 is thus greater than in the third illustration in FIG. 9.

As illustrated in FIG. 9, by means of the greater degree of filling, see the third and fourth illustrations for this purpose, the contact pressure of the cleaning component 6 against the housing 3 can be increased and better sealing made possible in order to achieve even more reliable cleaning.

The fifth illustration in FIG. 9 shows that the spatial extent 10 of the filled cleaning component 6 without the resistance of the housing 3 is greater than the spatial extent 11 of the housing 3.

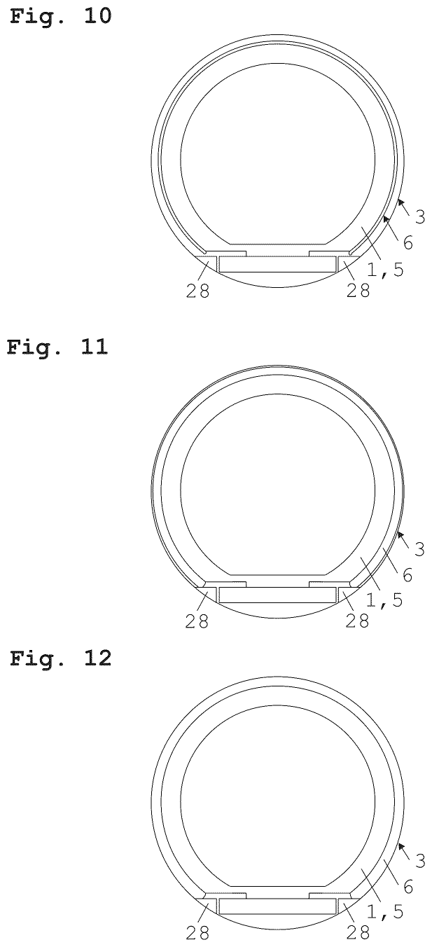

FIG. 10 shows a front view of a housing 3 with the cleaning unit 1 arranged therein from FIG. 3 in a non-filled state.

It can be seen in FIG. 10 that the cleaning component 6 is present in a non-filled state and therefore no contact with the housing 3 is realized.

FIG. 11 shows a front view of a housing 3 with the cleaning unit 1 from FIG. 3 arranged therein in a partially filled state shortly before a filled state.

It can be seen in FIG. 11 that the cleaning component 6 is already filled with a small amount of pressure medium, in comparison to FIG. 10, but the cleaning component 6, as before, is present in an only partially filled state, and therefore the housing 3 is not made contact with.

It should be noted at this juncture that, in FIG. 11, although the housing 3 is not contacted, the light strips 28 are contacted by the cleaning component 6.

FIG. 12 shows a front view of a housing with the cleaning unit from FIG. 3 arranged therein in a filled state.

It can be seen in FIG. 12 that, in comparison to FIGS. 10 and 11, the cleaning component 6 encloses a sufficient amount of pressure medium and is therefore in a filled state. In the illustrated filled state of the cleaning component 6, the housing 3 is contacted by the filled cleaning component 6.

As illustrated in FIG. 12, provision can preferably be made for the filled cleaning component 6 to realize gap-free or, in other words, continuous contact with the housing 3.

It should be noted at this juncture that, in FIG. 12, in addition to the housing 3, the light strips 28 are also contacted by the cleaning component 6. However, in this embodiment variant, the complete upper side of the light strips 27 is not contacted. In the event that the embodiment variant from FIG. 4 is used, in which the cleaning component 6 reaches as far as the beginning of the base part 27, there is the advantage of being able also to clean the complete upper side of the light strips 28 in addition to the housing 3. With the aid of the embodiment variant from FIG. 4, the cleaning component 6 also covers the gap, visible in FIGS. 10 to 12, present between the light strips 28 and the base part 27.

The following is a summary list of reference numerals and the corresponding structure used in the above description of the invention:

1 Cleaning unit
2 Patient-accommodating region

### 3 Housing

4 Medical imaging device
5 Support structure
6 Cleaning component
7 Contour, preferably outer contour, of the support structure
8 Outer contour of the cleaning component
9 Inner contour of the housing
10 Spatial extent of the cleaning unit
11 Spatial extent of the housing
12 Deformation device
13 Deformation drive
14 Connecting line
15 Cleaning main body
16 Cleaning covering
17 Outer contour of the cleaning main bodies
18 Displacement device
19 Patient table
20 Scanner unit
21 First direction
22 Second direction
23 Operating device
24 Output device
25 Displacement drive
26 Open-loop and closed-loop control unit of the displacement device
27 Base part
28 Light strip
29 Ring part
30 Coupling device

### 31 Connector

1. A cleaning unit for cleaning a housing of a medical imaging device that surrounds a patient-accommodating region, the cleaning unit comprising:
a support structure and a cleaning component;
when the cleaning unit is introduced into the housing, said cleaning component being arranged between said support structure and the housing; and
said cleaning component, in a pressure medium-filled state, resting against and contacting the housing.

2. A cleaning unit for cleaning a housing of a medical imaging device that surrounds a patient-accommodating region, the cleaning unit comprising:
a support structure and a cleaning component;
said cleaning component being arranged along an outer contour of said support structure; and
said cleaning component being deformable by filling with a pressure medium.

3. The cleaning unit according to claim 2, wherein at least one of an outer contour of said support structure or an outer contour of said cleaning component in a filled state is curved in correspondence with an inner contour of the housing.

4. The cleaning unit according to claim 1, wherein at least one of an outer contour of said support structure or an outer contour of said cleaning component in a filled state is curved in correspondence with an inner contour of the housing.

5. The cleaning unit according to claim 1, wherein at least one of the following is true:
a spatial extent of the cleaning unit is smaller when the cleaning component is not filled than the spatial extent of the cleaning unit when the cleaning component is filled; or
the spatial extent of the cleaning unit is smaller when the cleaning component is not filled than a spatial extent of the housing; or
the spatial extent of the cleaning unit is at least as large or larger when the cleaning component is filled than the spatial extent of the housing;
an external diameter of the cleaning unit is smaller when the cleaning component is not filled than an external diameter of the cleaning unit when the cleaning component is filled; or
the external diameter of the cleaning unit is smaller when the cleaning component is not filled than an internal diameter of the housing; or
the external diameter of the cleaning unit is at least as large or larger when the cleaning component is filled than an internal diameter of the housing.

6. The cleaning unit according to claim 2, wherein at least one of the following is true:
a spatial extent of the cleaning unit is smaller when the cleaning component is not filled than the spatial extent of the cleaning unit when the cleaning component is filled; or
the spatial extent of the cleaning unit is smaller when the cleaning component is not filled than a spatial extent of the housing; or
the spatial extent of the cleaning unit is at least as large or larger when the cleaning component is filled than the spatial extent of the housing;
an external diameter of the cleaning unit is smaller when the cleaning component is not filled than an external diameter of the cleaning unit when the cleaning component is filled; or
the external diameter of the cleaning unit is smaller when the cleaning component is not filled than an internal diameter of the housing; or
the external diameter of the cleaning unit is at least as large or larger when the cleaning component is filled than an internal diameter of the housing.

7. The cleaning unit according to claim 2, wherein the pressure medium is a gas or a liquid.

8. The cleaning unit according to claim 2, further comprising a deformation device having a deformation drive, a pressure medium, and a connecting line for connecting said cleaning component to said deformation drive, wherein said deformation drive is configured for selectively filling the pressure medium into said cleaning component or removing the pressure medium from said cleaning component, and wherein said cleaning component is deformable by filling or removing the pressure medium.

9. The cleaning unit according to claim 1, further comprising a deformation device having a deformation drive, a pressure medium, and a connecting line for connecting said cleaning component to said deformation drive, wherein said deformation drive is configured for selectively filling the pressure medium into said cleaning component or removing the pressure medium from said cleaning component, and wherein said cleaning component is deformable by filling or removing the pressure medium.

10. The cleaning unit according to claim 1, wherein said cleaning component has a cleaning main body and a cleaning covering.

11. The cleaning unit according to claim 10, further comprising a placement device configured for placing said cleaning covering onto said cleaning main body along an outer contour of said cleaning main body.

12. The cleaning unit according to claim 10, further comprising an application device for applying a cleaning product to or into said cleaning covering, wherein the cleaning product may be applied by way of said application device before, during, and/or after a placement of said cleaning covering onto said cleaning main body.

13. The cleaning unit according to claim 1, further comprising a displacement device for moving the cleaning unit within the medical imaging device, said displacement device being a movable patient table of the medical imaging device or a displacement device which is separate from the medical imaging device.

14. The cleaning unit according to claim 2, further comprising a displacement device for moving the cleaning unit within the medical imaging device, said displacement device being a movable patient table of the medical imaging device or a displacement device which is separate from the medical imaging device.

15. A medical imaging device, comprising:
a scanner unit, a patient-accommodating region at least partly surrounded by said scanner unit, a housing that surrounds the patient-accommodating region; and
at least one cleaning unit according to claim 1 configured for movement within said housing.

16. A medical imaging device, comprising:
a scanner unit, a patient-accommodating region at least partly surrounded by said scanner unit, a housing that surrounds the patient-accommodating region; and
at least one cleaning unit according to claim 2 configured for movement within said housing.

17. The medical imaging device according to claim 15, wherein:
when the cleaning component is not filled, the non-filled cleaning component is surrounded without contact by said housing; and
when the cleaning component is filled, the filled cleaning component rests against and contacts the housing.

18. The medical imaging device according to claim 15, wherein the cleaning unit is connected or connectable to a displacement device, the displacement device being a movable patient table of the medical imaging device or a displacement device separate from the medical imaging device.

19. A method for cleaning a medical imaging device, the method comprising:
providing the medical imaging device with a scanner unit, a patient-accommodating region at least partly surrounded by the scanner unit, and a housing that surrounds the patient-accommodating region; and
providing at least one cleaning unit according to claim 1 configured for movement within said housing;
providing the cleaning unit with a filled cleaning component; and
cleaning the housing of the medical imaging device with the filled cleaning component.

20. The method according to claim 19, which comprises:
moving the cleaning unit into the housing;
cleaning the housing by the filled cleaning component along at least one of a first direction or a second direction;
providing the cleaning unit with a non-filled cleaning component;
contactlessly moving the non-filled cleaning component within the housing, in at least one of a first direction or a second direction; and
moving the cleaning unit out of the housing.
